(19) United States
(12) Patent Application Publication
(10) Pub. No.: US 2025/0377334 A1
(43) Pub. Date: Dec. 11, 2025

(54) ADENO-ASSOCIATED VIRUS VECTORS EMPTY/FULL RATIO ANALYSIS USING CE-BASED GENOME AND CAPSID QUANTIFICATION

(71) Applicant: DH TECHNOLOGIES DEVELOPMENT PTE. LTD., Singapore (SG)

(72) Inventors: Elliott Jones, San Francisco, CA (US); Tingting Li, Yorba Linda, CA (US); Jane Luo, Irvine, CA (US); Sahana Mollah, Foster City, CA (US)

(21) Appl. No.: 18/876,307

(22) PCT Filed: Jul. 17, 2023
(86) PCT No.: PCT/IB2023/057276

§ 371 Date: Dec. 18, 2024

Related U.S. Application Data
(60) Provisional application No. 63/390,414, filed on Jul. 19, 2022.
(60) Provisional application No. 63/453,813, filed on Mar. 22, 2023.

(51) Int. Cl. G01N 27/447; C12Q 1/37; G01N 1/28; G01N 1/34; G01N 1/44; G01N 33/68

(52) U.S. Cl. CPC G01N 27/44747; C12Q 1/37; G01N 1/34; G01N 1/44; G01N 27/44726; G01N 33/6842; G01N 2001/2893; G01N 2333/015; G01N 2333/922; G01N 2333/96441

(57) ABSTRACT

The presently described and claimed disclosure relates to capillary electrophoresis methods for quantifying an intact AAV genome and protein components in an AAV using the same capillary electrophoresis system. The claimed and described approach offers an automated analysis of AAV samples and provides information to determine the AAV empty/full ratio.

### RELATED APPLICATIONS

The present patent application claims the priority benefit of U.S. Provisional Patent Application Ser. No. 63/390,414, filed Jul. 19, 2022 and U.S. Provisional Patent Application Ser. No. 63/453,813, filed Mar. 22, 2023, the content of each is hereby incorporated by reference in its entirety into this disclosure.

### BACKGROUND

There are various challenges during the manufacturing of an adeno-associated virus vector (AAV) or AAV-based gene products, including the ability to characterize the active virus product and capsids properly. Empty/full capsid ratio analysis is a critical quality attribute monitoring required for all viral vector-based gene product manufacturing. During the manufacturing process, the ratio of full and empty AAV capsids needs to be determined to achieve accurate therapeutic dosing and minimal potential immune response. AAV manufacturers and users do not have a fast, inexpensive test with low sample volume to determine the percent of full capsids in their products.

One conventional method often used for determining the percentage of the full genome in the total capsids is carried out by obtaining the number of genome vectors derived from the existing real-time polymerase chain reaction (qPCR) data and the total capsid number obtained from the enzyme-linked immunosorbent assay (ELISA) data. However, this method is limited by its insufficient data accuracy and precision. For example, qPCR is unable to differentiate between AAV full capsids (i.e., two ITR regions in an intact genome) and AAV partial capsids (e.g., one ITR, but the genome is not intact). Furthermore, the measurements require two different platforms. Another conventional method includes spectrophotometric-based methods that use the optical density of AAV samples at 260 nm and 280 nm to determine the protein and DNA content in the samples. However, this method requires high purity of the AAV sample to minimize the interference of the impurities with UV absorbance at 260 nm and 280 nm and has low accuracy.

Gradient ultra-centrifugation is often used to purify packaged rAAV vectors (Full capsids) from cellular debris contaminants, host cell DNA, RNA, and Empty AAV capsids. However, remnants of contaminants can still be present in the viral vector product after purification. While transmission electron microscopy (TEM) could count the full and empty particles as a population, it is very difficult to distinguish the partial capsids, and it is too time-consuming for data analysis. Although analytical ultracentrifugation (AUC) can separate full capsids from empty capsids, it is resource intensive as AUC instruments are expensive and require large amounts of samples for analysis, it is impractical to use AUC for QC release tests.

### SUMMARY

As conventional methodologies are often inaccurate, complex, impractical, and/or require a large amount of samples, the need exists for a suitable method for manufacturing process development and/or routine analysis of AAV. The inventors have recognized the need for capillary electrophoresis methods for quantifying an intact AAV genome and protein components in an AAV using the same capillary electrophoresis system. The claimed and described approach offers an automated analysis of AAV samples and provides information to determine the AAV empty/full ratio.

One aspect of the disclosure relates to a method of evaluating at least one adeno-associated virus vector (AAV) sample, the method including loading a first portion of the AAV sample on a first capillary electrophoresis (CE) capillary, wherein the first CE capillary is filled with a first buffer comprising a first polymer matrix; applying a voltage to the first AAV portion to separate an intact AAV genome from the first portion of the AAV sample; detecting the separated AAV genome with a detector; producing an electropherogram including corrected peak area of the intact AAV genome and generating a first corresponding set of values; loading a second portion of the AAV sample on a second capillary electrophoresis (CE) capillary, wherein the second CE capillary is filled with a second buffer including sodium dodecyl sulfate (SDS) and a second polymer matrix; applying a voltage to the second portion of the AAV sample to separate an AAV capsid protein component from the second portion of the AAV sample; detecting the separated AAV capsid protein component with a detector; producing an electropherogram including corrected peak area of the AAV capsid protein component and generating a second corresponding set of values; and wherein the first corresponding set of values is used to quantify the intact AAV genome in the AAV sample and the second corresponding set of values is used to quantify the AAV capsid protein component in the AAV sample.

In an aspect, the AAV sample is selected from the group including wild-type AAVs, recombinant AAVs, AAV serotypes, self-complementary AAVs, and AAV drug products. In another aspect, the AAV capsid protein component is selected from the group including VP1, VP2, VP3, and combinations thereof.

In an aspect, the intact AAV genome is separated from partial genomes and/or impurities in the first portion of the AAV sample. In another aspect, the AAV capsid protein component is separated from impurities in the second portion of the AAV sample. In an aspect, the AAV capsid protein component is VP3.

In an aspect, the intact AAV genome concentration is determined by comparing the first corresponding set of values with an intact AAV genome calibration standard. In an aspect, the intact AAV genome calibration standard is generated by loading a AAV genome standard series on a capillary electrophoresis (CE) capillary, wherein the CE capillary is filled with a first buffer comprising a first polymer matrix, the AAV genome standard series including at least one concentration of an AAV genome standard; applying a voltage to the AAV genome standard series to separate intact genome from the AAV genome standard; detecting the separated intact genome with a detector; producing an electropherogram including corrected peak area of the intact genome and generating a corresponding set of AAV genome standard values; and wherein the intact AAV genome calibration standard is generated from the corresponding set of AAV genome standard values.

In another aspect, the AAV genome standard series includes an AAV sample with a known titer, an RNA sample with known concentration, and/or a single stranded DNA sample with known concentration. In an aspect, the AAV genome standard series includes an RNA sample with a known concentration, and wherein the concentration of the RNA sample is further correlated to an intact genome titer of an AAV genome standard. In an aspect, the AAV genome standard series including an AAV sample with the known titer and known full %. In another aspect, the known full % is obtained using AUC. In an aspect, the standard series includes an AAV sample with the known titer equal or higher than 1×1013 GC/ml. In another aspect, the known full % of the AAV genome standard is determined using an AAV reference material with full % determined by AUC and with a known titer. In an aspect, the concentration of the intact AAV genome is determined by comparing the first corresponding set of values with the intact AAV genome calibration standard and further correcting to account for a presence of genome other than intact genome in the AAV genome standard series. In an aspect, the method further includes correcting to account for a presence of genome other than intact genome in the AAV genome standard series comprises multiplying by full % determined by AUC.

In an aspect, the concentration of the AAV capsid protein component is determined by comparing the second corresponding set of values with an AAV capsid protein calibration standard. In an aspect, the AAV capsid protein calibration standard is generated by loading a AAV capsid protein standard series on a second capillary electrophoresis (CE) capillary, wherein the second CE capillary is filled with a second buffer including sodium dodecyl sulfate (SDS) and a second polymer matrix, the AAV capsid protein standard series comprising at least one concentration of an AAV capsid protein standard; applying a voltage to the second standard series to separate AAV capsid protein components from AAV capsid protein standard; detecting the separated AAV capsid protein components with a detector; and producing an electropherogram including corrected peak area of the AAV capsid protein component; generating a corresponding set of AAV capsid standard values and wherein the AAV capsid protein calibration standard is generated from the corresponding set of AAV capsid standard values. In an aspect, the AAV capsid protein standard series includes an AAV sample with a known concentration, an AAV sample with a known titer, and/or a protein sample with a known concentration. In another aspect, the AAV capsid protein standard series includes an AAV capsid standard sample with a known titer and known full %. In an aspect, the known full % is obtained using AUC. In another aspect, the known full % of the AAV capsid standard is determined using an AAV reference material with known full % by AUC and with a known titer. In an aspect, the AAV capsid protein standard series comprises at least two different concentration of an AAV capsid protein standard.

In an aspect, the AAV genome standard and the AAV capsid protein standard are the same.

In an aspect, the first corresponding set of values is divided by the second of corresponding values to determine a percentage of full AAV capsids with intact AAV genomes. In an aspect, the first corresponding set of values is divided by the second of corresponding values and multiplied by the percent of intact genome of the AAV standard to determine a percentage of full AAV capsids with intact AAV genomes in a test sample. In another aspect, the method further includes correcting to account for a presence of a genome other than intact genome in the AAV genome standard series comprises multiplying by full % determined by AUC. In an aspect, an empty/full ratio of the AAV sample is calculated using the full %.

In an aspect, first CE capillary is housed in a first capillary cartridge and the second CE capillary is housed in a second capillary cartridge. In an aspect, the first buffer including a first polymer matrix is different from the second buffer including a second polymer matrix. In an aspect, the first polymer or second polymer matrix is independently selected from the group including crosslinked polymer, linear polymers, slightly branched polymers, linear polyacrylamide, polyvinylpyrrolidone, polyethylene oxide, polyethylene glycol and dextran.

In an aspect, the first portion of the AAV sample is denatured or digested prior to loading onto the first CE capillary. In an aspect, the first portion of the AAV sample is denatured prior to CE separation. In another aspect, the first portion of the AAV sample is digested using at least one endonuclease, protease, peptidase or proteinase. In an aspect, the endonuclease is selected from the group consisting of DNase I, benzonase, RNase, and combinations thereof. In an aspect, the protease, peptidase or proteinase is Proteinase K.

In an aspect, the first portion of the AAV sample is purified or enriched prior to loading onto the first CE capillary. In another aspect, the first portion of the AAV sample is purified or enriched using spin columns, spin tubes, and/or magnetic beads. In an aspect, the first portion of the AAV sample is diluted with a sample solution, water, or combinations thereof prior to loading on the CE capillary.

In an aspect, the method further includes heating the first portion of the AAV sample prior to loading the first portion of the AAV sample on the CE capillary. In an aspect, the first portion of the AAV sample is heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C., alternatively at a temperature of about 60° C. In another aspect, the first portion of the AAV sample is heated at a temperature of about 70° C. In an aspect, the first portion of the AAV sample is heated for at least 2 minutes, alternatively at least 3 minutes, alternatively at least 4 minutes, alternatively at least 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes. In another aspect, the first portion of the AAV sample is heated for at least about 2 minutes.

In an aspect, the method further includes cooling the first portion of the AAV sample after heating. In an aspect, the first portion of the AAV sample is cooled using ice or at about 4° C. In an aspect, the first portion of the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

In an aspect, the method further includes adding a fluorescent dye to the first buffer comprising a first polymer matrix, wherein the fluorescent dye binds the AAV sample resulting in fluorescently labeled AAV genome. In an aspect, the fluorescent dye is a cyanine-based dye. In an aspect, the fluorescent dye is selected from the group including Cy2, Cy3, Cy3B, Cy3.5, Cy5, Cy5.5, Cy7, SYBR Green I, SYBR GOLD, SYBR Green II, PicoGreen, Thiazole orange, and Oxazole yellow.

In an aspect, the second portion of the AAV sample is denatured or digested prior to loading onto the second CE capillary. In another aspect, the second portion of the AAV sample is denatured prior to CE separation. In an aspect, the second portion of the AAV sample is denatured using heat, detergent, a reducing agent, sonication, or a combination thereof. In another aspect, the second portion of the AAV sample is denatured using heat, SDS, and dithiothreitol. In an aspect, the second portion of the AAV sample is heated at a temperature of about alternatively about 50° C., alternatively about 55° C., alternatively about 60° C., alternatively about 65° C., alternatively about 70° C., alternatively about 75° C., alternatively about 80° C., alternatively about 85° C., alternatively about 90° C., alternatively about 95° C. In another aspect, the second portion of the AAV sample is heated at a temperature of about 70° C. In an aspect, the second portion of the AAV sample is heated for at least about 5 minutes, alternatively at least about 10 minutes, alternatively at least about 15 minutes, alternatively at least about 20 minutes, alternatively at least about 25 minutes, alternatively at least about 30 minutes, alternatively at least about 45 minutes, alternatively at least about 60 minutes. In another aspect, the second portion of the AAV sample is heated for at least about 10 minutes.

In an aspect, the second portion of the AAV sample is cooled to room temperature. In another aspect, the second portion of the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

In an aspect, the method further includes adding a fluorescent dye to the denatured second portion of the AAV sample, wherein the fluorescent dye reacts the AAV sample resulting in fluorescently labeled AAV protein component. In an aspect, the fluorescent dye is incubated with the denatured second portion of the AAV sample at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C. In another aspect, the fluorescent dye is incubated with the denatured second portion of the AAV sample at a temperature of about 70° C. In an aspect, the fluorescent dye is incubated with the denatured second portion of the AAV sample for at least about 5 minutes, alternatively at least about 10 minutes, alternatively at least about 15 minutes, alternatively at least about 20 minutes, alternatively at least about 25 minutes, alternatively at least about 30 minutes, alternatively at least about 45 minutes, alternatively at least about 60 minutes. In another aspect, the fluorescent dye is incubated with the denatured second portion of the AAV sample for at least about 10 minutes. In an aspect, the fluorescent dye is a cyanine-based dye or a pyrylium-based dye. In another aspect, the fluorescent dye is selected from the group consisting of Cy2, Cy3, Cy3B, Cy3.5, Cy5, Cy5.5, Cy7, Rhodmine, Fluorescein, and Fluorescent Chromeo™ Py-Dyes.

In an aspect, the method further includes cooling the second portion of the AAV sample to room temperature after heating. In an aspect, the second portion of the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

In an aspect, the second portion of the AAV sample is diluted with a sample solution, water, or combinations thereof prior to loading on the second CE capillary.

In an aspect, the detector is a UV detector or fluorescence detector. In another aspect, the detector is a laser-induced fluorescence (LIF) detector, a lamp-based fluorescence detector, or a native fluorescence detector. In an aspect, when the separated AAV genome is detected, the detector is set at an excitation wavelength of about 488 nm and an emission wavelength of about 520 nm, and when the separated AAV protein component is detected, the detector is set at an excitation wavelength of about 488 nm and an emission wavelength of about 600 nm.

In an aspect, the method is used in a high-throughput application or a rapid analysis workflow.

One aspect of the disclosure provides for a kit for quantifying an intact adeno-associated virus vector (AAV) genome and quantifying an AAV protein component, the kit including: a first buffer including a first polymer matrix, a second buffer including sodium dodecyl sulfate (SDS) and a second polymer matrix, and instructions for use. In an aspect, the kit further includes at least two capillary electrophoresis (CE) capillaries or at least two CE cartridge comprising at least one capillary. In another aspect, the kit further includes at least one fluorescent dye that binds nucleic acids, at least one fluorescent dye that labels protein, a diluent, a nuclease and/or a proteinase/protease.

Other aspects and features of the present disclosure will become apparent to those ordinarily skilled in the art upon review of the following description of specific embodiments of the disclosure in conjunction with the accompanying figures.

### BRIEF DESCRIPTION OF THE FIGURES

Embodiments of the present disclosure will now be described, by way of example only, with reference to the attached Figures.

FIG. 1A is a pictorial representation of an adeno-associated virus.

FIG. 1B is a pictorial representation of AAV full capsids and AAV partial capsids.

FIG. 2A shows electropherogram results for an intact AAV genome peak separated from the partial genome and other impurities in AAV samples.

FIG. 2B shows an AAV genome calibration standard curve of corrected peak area of intact genome peak vs. AAV concentration or titer.

FIG. 3A shows electropherogram results for AAV capsid protein VP3 separated from other capsid proteins and impurities in AAV samples.

FIG. 3B shows an AAV capsid protein calibration standard curve of the corrected peak area of VP3 vs. AAV concentration or titer.

FIGS. 4A and 4B show various scenarios for the determination of full % of an AAV test sample according to an aspect of this disclosure.

FIG. 5 shows a calibration standard curve made using a 1.8 kb firefly luciferase (Fluc) mRNA standard by plotting the corrected peak area of the Fluc mRNA peak against Fluc mRNA concentration.

FIG. 6 shows an AAV genome calibration standard curve of corrected peak area of intact genome peak vs. AAV concentration or titer. Samples for this standard curve were separated in the same sequence as for the samples in FIG. 5 and using the same separation conditions (same separation method, same cartridge, and same matrix). The corrected peak area of the Fluc mRNA at each concentration in FIG. 5 was converted to AAV concentration or titer using the linear equation from FIG. 6.

FIG. 7 shows an AAV genome calibration standard curve with AAV titer (converted from Fluc mRNA CPA) vs mRNA concentration.

### DETAILED DESCRIPTION

It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a”, “an”, and “the” include plural reference unless the context clearly indicates otherwise. The singular form “a”, “an”, and “the” include plural referents unless the context clearly dictates otherwise. These articles refer to one or to more than one (i.e., to at least one). The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

The term “about” is used in connection with a numerical value throughout the specification and the claims denote an interval of accuracy, familiar and acceptable to a person skilled in the art. In general, such an interval of accuracy is +/−10%.

The term “exemplary” means serving as a non-limiting example, instance, or illustration. As utilized herein, the terms “e.g.,” and “for example” set off lists of one or more non-limiting aspects, examples, instances, or illustrations.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Unless defined otherwise, all technical and scientific terms used herein have the same meaning as commonly understood to one of ordinary skill in the art to which this disclosure belongs.

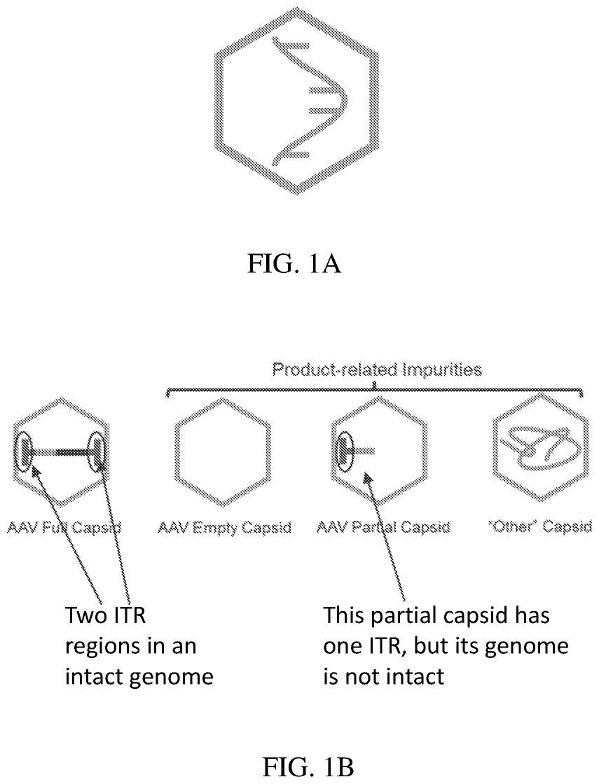

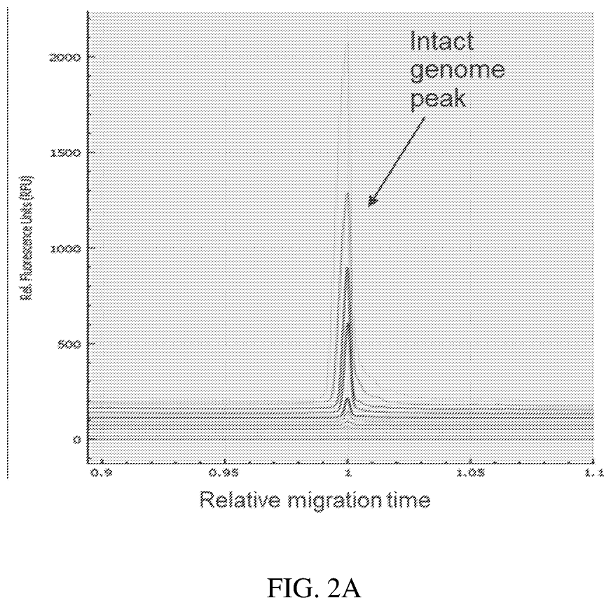

Adeno-associated virus (AAV) (FIG. 1A) is a small (25-nm) virus that is composed of a non-enveloped icosahedral protein shell called capsid and a single-stranded DNA genome of about 4.7 kb. With its excellent safety profile and high efficiency in transducing a broad range of target tissues, the AAV vector has become an attractive choice for gene therapy.

So far, AAV exists in some 13 human and primate serotypes which, in combination with the primary sequence differences, mediate the AAV cell and tissue specificity. For example, AAV 8 or serotype 8 is efficient in transducing hepatocytes. This structure has the ability to carry up to 5 Kb of pay-load of single stranded DNA molecule. The capsid or viral proteins are translated from the same mRNA encoding overlapping sequences of three capsid proteins, VP1, VP2, and VP3, with molecular weights of approximately 87, 72, and 62 kDa, respectively. Each AAV capsid is composed of 60 monomers of VP1, VP2, and VP3 in a ratio of 1:1:10.

Typically, AAV concentration used in gene therapy is in the order of 1×10¹⁰ GC/mL (GC=genomic copies), which equates to 50 ng/mL and thus falls well below the limit of detection or quantitation of any UV absorbance based assays.

Two recombinant AAV (rAAV) based drugs have been approved by the FDA: Zolgensma by AveXis for spinal muscular atrophy and Luxturna by Spark Therapeutics for inherited blindness. Many more are in clinical trials. One of the most commonly used methods for the production of rAAV vectors is the triple-transfection method which involves co-transfection of permissive cells such as HEK293 cells with three plasmids: one containing the transgene of interest flanked by the AAV inverted terminal repeats (ITRs), a packaging plasmid containing rep and cap genes, and a third plasmid encoding adenoviral helper genes.

The quality of full capsids directly impacts the efficacy of the treatment, including the outcome of both preclinical and clinical studies. Therefore, it is crucial to accurately assess the quality and the correct length/size of the genome encapsidated in the vector. Conventional methods, for example, qPCR that targets the ITR region, are unable to differentiate between AAV full capsids with intact genome (i.e., with two ITR regions in an intact genome) and AAV partial capsids (e.g., one ITR, but the genome is not intact). (e.g., FIG. 1B).

Aspects of this disclosure include methods of evaluating an adeno-associated virus vector (AAV) sample using a capillary electrophoresis platform. In some aspects, using the same capillary electrophoresis platform, methods of this disclosure can be used to determine the ratio of AAV genome quantitation and total capsid/protein quantitation to determine the AAV empty/full ratio. Additionally, these methods can provide purity analysis of the viral vector and genome integrity of the viral vector.

An aspect of this disclosure includes methods for quantifying the intact genome content in an AAV sample and quantifying protein component(s) in the same AAV sample using a single capillary electrophoresis platform. The AAV sample may be a wild-type AAV, recombinant AAV, AAV serotype, self-complementary AAV, or AAV drug product. Depending on the analysis desired, one or more different AAV serotypes may be evaluated.

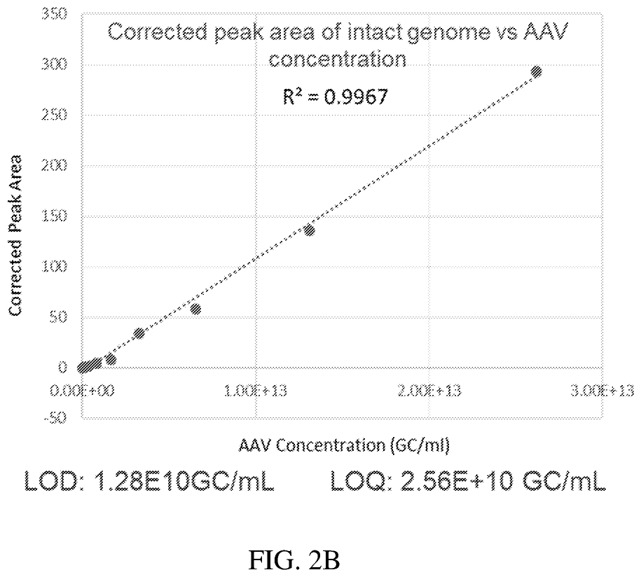

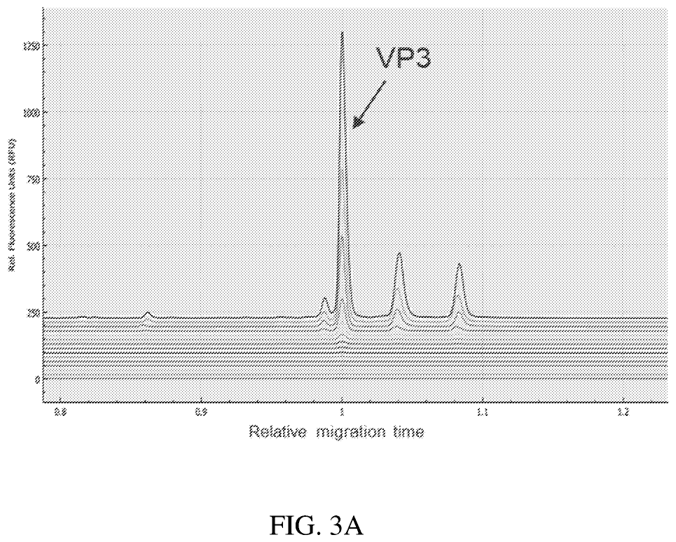

In an aspect, this method includes loading a first portion of an AAV sample on a first capillary electrophoresis (CE) capillary, wherein the first CE capillary is filled with a first buffer comprising a first polymer matrix; applying a voltage to the first AAV portion to separate an intact AAV genome from the partial genome and other impurities in the first portion of the AAV sample and detecting the separated intact AAV genome with a detector, thereby producing an electropherogram from which the corrected peak area of the intact AAV genome peak is determined. FIG. 2A shows electropherogram results for an intact AAV genome peak separated from the partial genome and other impurities in AAV samples. In an aspect, a first corresponding set of values may include the corrected peak area of the intact AAV genome peak.

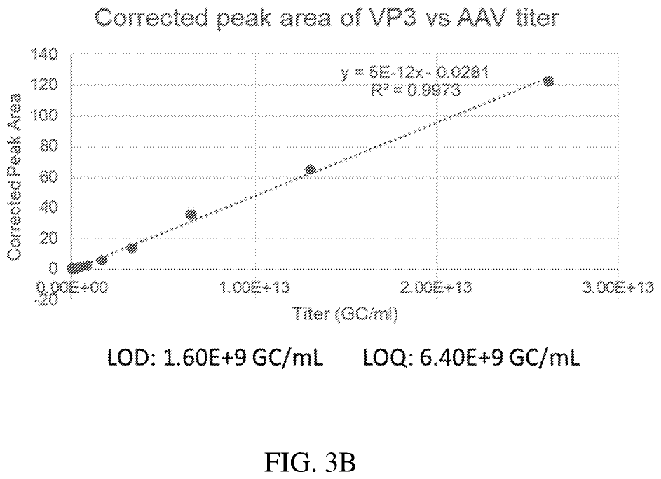

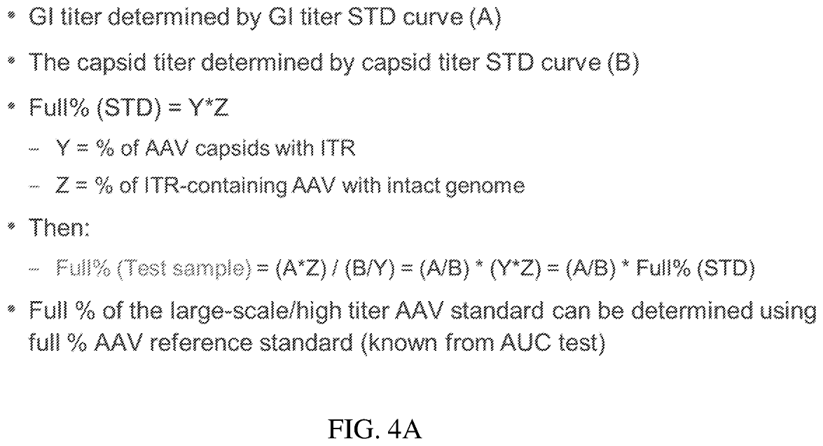

In an aspect, the method also includes loading a second portion of the AAV sample on a second capillary electrophoresis (CE) capillary, wherein the second CE capillary is filled with a second buffer comprising sodium dodecyl sulfate (SDS) and a second polymer matrix; applying a voltage to the second portion of the AAV sample to separate an AAV capsid protein component from impurities in the second portion of the AAV sample; detecting the separated AAV capsid protein component with a detector, thereby producing an electropherogram from which the corrected peak area of the capsid peak, for example VP1, VP2, VP3 or combinations thereof, is determined. FIG. 3A shows electropherogram results for AAV capsid protein VP3 separated from other capsid proteins and impurities in AAV samples. In an aspect, a second corresponding set of values may include the corrected peak area of the capsid peak.

In an aspect, the concentration of the intact AAV genome may be determined by comparing the first corresponding set of values with an intact AAV genome calibration standard. In a non-limiting example, the intact AAV genome calibration standard may be generated by loading an AAV genome standard series on a capillary electrophoresis (CE) capillary, wherein the CE capillary is filled with a first buffer comprising a first polymer matrix, the AAV genome standard series comprising at least one concentration of an AAV genome standard. In some aspects, the AAV genome standard series comprises at least two different concentration of an AAV genome standard.

In this aspect, the method further includes applying a voltage to the AAV genome standard series to separate intact AAV genomes from the AAV genome standards series; detecting the separated AAV genomes with a detector; producing an electropherogram comprising the corrected peak area of the intact AAV genome and generating a corresponding set of AAV genome standard values; wherein an intact AAV genome calibration standard is generated from the corresponding set of AAV genome standard values. FIG. 2B shows an AAV genome calibration standard of corrected peak area of intact genome peak vs. AAV concentration or titer.

In another aspect, the concentration of the AAV capsid protein component may be determined by comparing the second corresponding set of values with an AAV capsid protein calibration standard. In a non-limiting example, the AAV capsid protein calibration standard may be generated by loading an AAV capsid protein standard series on a second capillary electrophoresis (CE) capillary, wherein the second CE capillary is filled with a second buffer comprising sodium dodecyl sulfate (SDS) and a second polymer matrix, the AAV capsid protein standard series comprising at least one concentration of an AAV capsid protein standard. In some aspects, the AAV capsid protein standard series comprises at least two different concentrations of an AAV capsid protein standard.

In this aspect, the method further includes applying a voltage to the AAV capsid protein standard series to separate AAV capsid protein components from the AAV capsid protein standards; detecting the separated AAV capsid protein components with a detector; producing an electropherogram comprising the corrected peak area of the AAV capsid protein component and generating a corresponding set of AAV capsid standard values; wherein an AAV capsid protein component calibration standard is generated from the corresponding set of AAV capsid standard values. FIG. 3B shows an AAV capsid protein calibration standard of the corrected peak area of VP3 vs. AAV titer.

The different concentrations of the AAV genome standard and/or AAV capsid protein standard may be, for example, generated by dilutions or provided as AAV standards. A non-limiting example of a dilution series can be 1/1, 1/2, 1/5, 1/10, 1/15, 1/20, 1/50, 1/100, 1/200. When generating an intact AAV genome calibration standard, the AAV genome standard may be an AAV sample with a known concentration, an AAV sample with a known titer, RNA with a known concentration and/or a single stranded DNA standard with a known concentration. When generating an AAV capsid protein component calibration standard, the AAV capsid protein standard may be an AAV sample with a known concentration, an AAV sample with a known titer and/or a protein standard. In some non-limiting examples, the AAV genome standard and the AAV capsid protein standard are the same.

In an aspect, these first set and second set of corresponding values may be plotted as a single point, linear curves, in a tabular format, or using spreadsheet software. Once plotted, the intact AAV genome in an AAV test sample and the amount of protein components in the same AAV test sample can be determined. Once these values are determined, they can be used to obtain a percentage of full capsids with intact genome to determine the full % for an AAV sample.

For example, full % for an AAV sample can be determined using the following formula: Full % (Test)=(A/B)*Full % (STD), wherein A can correspond to an intact genome present (e.g., quantity, concentration), B can correspond to the AAV capsid protein component (e.g., quantity, concentration), and full % (STD) can correspond to full % in an AAV sample with known titer. In an example, A (or Value A) can be determined using a standard curve built with AAV samples of known titer or concentrations for genome integrity analysis, B (or Value B) can be determined using a standard curve built with AAV samples of known concentrations for capsid protein analysis. In some examples, the empty/full AAV capsid ratio can be determined using a similar approach (e.g., empty/full ratio=(100%−full %−partial %)/full % or empty/full ratio=(100%−full %)/full %).

In some aspects, the accuracy of the determination of full % capsids in a sample might depend on the characterization data available for the standards used for such determination. For example, when an AAV sample with known titer is used, the known titer might have been determined using techniques that do not (e.g., not fully or not accurately) take into account the presence of partial genomes or other species. For example, some methods based on the presence of the ITR regions (e.g., qPCR) might account for the presence of capsids with partial genome that still have ITR regions or other such species as a full capsid. And, as an example, might overestimate the genome titer. And, for example, the full % capsids in a sample might not be accurate if determined using such an AAV sample with known titer, unless an adjustment is made to account for the presence of genomes other than the intact genome (e.g., such as partial genomes). In another example, when an AAV sample with known titer is used, the known titer might have been determined using techniques that do take into account the presence of genomes other than the intact genome, such as partial genomes or other species. For example, analytical ultracentrifugation (AUC) might be used as such a technique. And, for example, the full % capsids in a sample might be accurate if determined using such an AAV sample with known titer and known full % capsids by AUC, without further adjustments to account for the presence of genomes other than the intact genome (e.g., such as partial genomes in capsids).

In some aspects, high-titer AAV samples are evaluated using the disclosed methods. In these situations, there might not be an AAV standard with a known titer high enough to accurately produce an intact AAV genome calibration standard or an AAV capsid protein calibration standard. In an aspect, a high-titer AAV sample with known titer is used as a standard for generating both an intact AAV genome calibration standard and an AAV capsid protein calibration standard. In an example, the full % of such samples might not have been determined using good techniques, such as AUC, that do take into the account the presence of genomes other than the intact genome, such as partial genomes or other species. In this aspect, the titer of this high-titer AAV sample may be determined by qPCR test using primers targeted to the ITR region. In an example, the full % by AUC might not be available for the high-titer AAV sample. But such value (or a similar determination) would be desired to calculate the full % of the AAV test samples. To address this problem, AAV reference materials (i.e., AAVs with both known titer and full % determined by AUC) are used to determine the full % for the high-titer AAV standard. The AAV reference material is available in various serotypes including, but not limited to, AAV1, AAV2, AAV5, AAV6, AAV8, and AAV9 serotypes. As the full % of the AAV reference materials is known, it can be used to characterize or quantify the full % of the high-titer AAV sample.

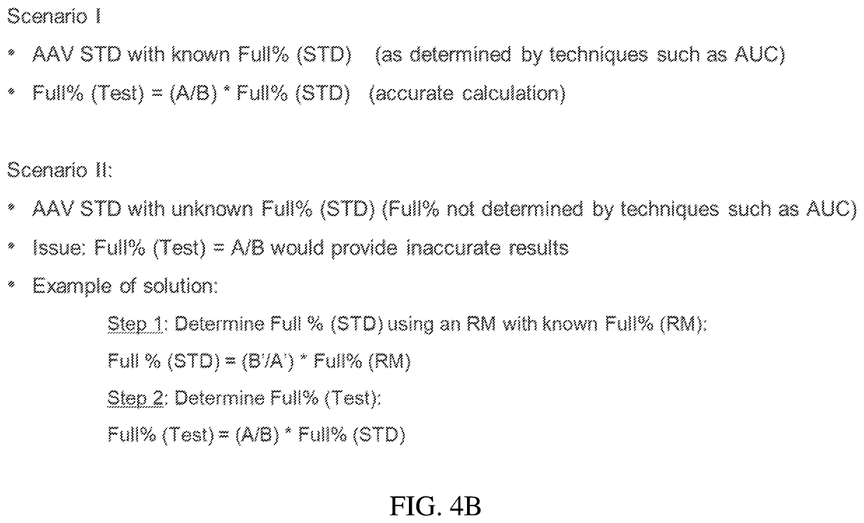

In this aspect, the AAV reference material is used as the test sample and the high-titer AAV sample with known titer is used as the standard. The full % of the high-titer AAV sample (i.e., STD) can be calculated because the full % for AAV reference material (i.e., RM) is known from an AUC test. Assuming that Full % (STD)=Y*Z, wherein Y=% of AAV capsids with ITR and Z=% of ITR-containing AAV with intact genome, the following calculation can be used to determine the Full % (STD). For example: Full % (STD)=(B′/A′)*Full % (RM), wherein B′ is capsid titer of the AAV reference material determined using the high titer standard and A′ is intact genome titer of the AAV reference material determined using the high titer standard. Then the full % of the test sample can be determined using Full % (Test)=(A/B)*Full % (STD), (See FIGS. 4A and 4B).

As used herein, “capillary” refers to a channel, tube, or other structure capable of supporting a volume of separation medium for performing electrophoresis. Capillary geometry can vary and includes structures having circular, rectangular, or square cross-sections, channels, grooves, plates, and the like that can be fabricated by technologies known in the art. Capillaries of the present disclosure can be made of materials such as, but not limited to, silica, fused silica, quartz, silicate-based glass such as borosilicate glass, phosphate glass, or alumina-containing glass, and other silica-like materials. In some aspects, the methods can be adapted and used in any generally known electrophoresis platform, such as, for example, electrophoresis devices comprising single or multiple microfluidic channels, etched microfluidic capillaries, as well as slab gel and thin-plate gel electrophoresis.

The first and/or second CE capillary may be housed in a capillary cartridge. In a non-limiting example, the capillary cartridge may be pre-assembled.

CE capillary may be filled with a buffer comprising a polymer matrix or gel buffer prior to applying a separation voltage and/or loading the AAV sample. In some aspects, the first buffer comprising a first polymer matrix is different from the second buffer comprising a second polymer matrix. In some aspects, a buffer comprising a polymer matrix or gel buffer is placed into a buffer vial. These buffer vials may be placed into buffer trays. In some aspects, a buffer comprising a polymer matrix or gel buffer may comprise additional components to facilitate the separation of the components of interest. Non-limiting examples of a suitable polymer matrix include crosslinked polymer, linear polymers, slightly branched polymers, linear polyacrylamide, polyvinylpyrrolidone, polyethylene oxide, polyethylene glycol, and dextran.

In an aspect, the first portion of the AAV sample is denatured or digested prior to loading onto the first CE capillary. In a non-limiting example, the first portion of the AAV sample is also denatured prior to CE separation. The AAV sample may be digested using at least one endonuclease, protease, peptidase, proteinase, or a combination thereof. In a non-limiting example, the endonuclease is DNase I, benzonase, RNase, and combinations thereof, and the protease, peptidase, or proteinase is Proteinase K.

In an aspect, the first portion of the AAV sample may be purified or enriched prior to loading onto the first CE capillary by using, for example, spin columns, spin tubes, and/or magnetic beads. Prior to loading the first portion of the AAV sample onto the CE capillary, in some aspects, the first portion of the AAV sample is diluted with a sample solution, water, or combinations thereof.

In an aspect, the method may further include heating the first portion of the AAV sample prior to loading the first portion of the AAV sample on the first CE capillary. In various aspects, the first portion of the AAV sample is heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C., alternatively at a temperature of about 60° C. In a non-limiting example, the first portion of the AAV sample is heated at a temperature of about 70° C.

In various aspects, the first portion of the AAV sample is heated for at least 2 minutes, alternatively at least 3 minutes, alternatively at least 4 minutes, alternatively at least 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes. In a non-limiting example, the first portion of the AAV sample is heated for at least about 2 minutes.

In an aspect, the method may further include cooling the first portion of the AAV sample on ice or at 4° C. after heating. Rapid or quick cooling of the AAV sample on ice or at 4° C. ensures that the AAV samples stay in an unfolded position. In a non-limiting example, the first portion of the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

An aspect of the method may also include adding a detectable dye to the first buffer comprising a first polymer matrix, wherein the detectable dye binds the AAV sample resulting in a detectable dye-labeled AAV genome. Another aspect of the method may also include separately adding a detectable dye to the denatured second portion of the AAV sample, wherein the detectable dye reacts with the AAV sample resulting in a detectable dye-labeled AAV capsid protein component. The detectable dye may be incubated with the second portion of the AAV sample for a time and/or temperature sufficient to form a labeled capsid protein component.

In an aspect, the method may include denaturing or digesting the second portion of the AAV sample prior to loading it onto the second CE capillary. In some aspects, the second portion of the AAV sample is denatured prior to CE separation. In a non-limiting example, the second portion of the AAV sample is denatured using heat, detergent, a reducing agent and/or sonication, or a combination thereof. For example, the second portion of the AAV sample may be denatured using heat, SDS (a detergent), and dithiothreitol (a reducing agent)

If the second portion of the AAV sample is denatured using heat, they may be heated at a temperature of about alternatively about 50° C., alternatively about 55° C., alternatively about 60° C., alternatively about 65° C., alternatively about 70° C., alternatively about 75° C., alternatively about 80° C., alternatively about 85° C., alternatively about 90° C., alternatively about 95° C. In a non-limiting example, the second portion of the AAV sample is heated at a temperature of about 70° C.

In an aspect, the second portion of the AAV sample is heated for at least about 5 minutes, alternatively at least about 10 minutes, alternatively at least about 15 minutes, alternatively at least about 20 minutes, alternatively at least about 25 minutes, alternatively at least about 30 minutes, alternatively at least about 45 minutes, alternatively at least about 60 minutes. In a non-limiting example, the second portion of the AAV sample is heated for at least about 10 minutes.

After heating, in an aspect, the second portion of the AAV is cooled to room temperature.

In a non-limiting example, the detectable dye may be incubated with the denatured second portion of the AAV sample at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C. The detectable dye may be incubated with the denatured second portion of the AAV sample for at least about 5 minutes, alternatively at least about 10 minutes, alternatively at least about 15 minutes, alternatively at least about 20 minutes, alternatively at least about 25 minutes, alternatively at least about 30 minutes, alternatively at least about 45 minutes, alternatively at least about 60 minutes.

In another non-limiting example, after incubation of the detectable dye, the second portion of the AAV sample is cooled to room temperature. For example, the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

The second portion of the AAV sample may then be diluted with a sample solution, water, or combinations thereof prior to loading on the second CE capillary.

The detectable dye may be a fluorescent dye and includes dyes that independently have an absorption wavelength and emission wavelength of between about 480 nm and about 740 nm, alternatively about 490 nm, alternatively about 500 nm, alternatively about 510 nm, alternatively about 520 nm, alternatively about 530 nm, alternatively about 540 nm, alternatively about 550 nm, alternatively about 560 nm, alternatively about 570 nm, alternatively about 580 nm, alternatively about 590 nm, alternatively about 600 nm, alternatively about 610 nm, alternatively about 620 nm, alternatively about 630 nm, alternatively about 640 nm, alternatively about 650 nm, alternatively about 660 nm, alternatively about 670 nm, alternatively about 680 nm, alternatively about 690 nm, alternatively about 700 nm, alternatively about 710 nm, alternatively about 720 nm, alternatively about 730 nm.

Non-limiting examples of the detectable dye include cyanine-based dyes and pyrylium-based dyes. Cyanine-based dyes are characterized by a quaternary nitrogen and a tertiary nitrogen joined together by a chain of several conjugated carbon atoms. Pyrylium-based dyes change colors and become fluorescent upon reacting with primary amines. Non-limiting examples of suitable cyanine-based dyes include Cy2, Cy3, Cy3B, Cy3.5, Cy5, Cy5.5, and Cy7 and Cy7. Non-limiting examples of suitable pyrylium-based dyes include Fluorescent Chromeo™ Py-Dyes. When labeling the first portion of the AAV sample, detectable dyes such as SYBR Green I, SYBR Green II, SYBR GOLD, PicoGreen, Thiazole orange, and Oxazole yellow may be used.

In an aspect of the method, the separated AAV genome and/or separated AAV capsid protein component are detected using a detector. The detector can be a UV or fluorescence detector, such as a laser induced fluorescence (LIF) detector or a lamp-based fluorescence detector. In some aspects, when the separated AAV capsid protein component is being detected, a native fluorescence detector may be used. The desired quantitation sensitivity will determine the type of detector used. LIF detection offers the benefit of about a 100-fold increase in sensitivity, yet it also requires additional sample manipulation.

In order to detect both the separated AAV genome and separated AAV capsid protein component on the same capillary electrophoresis system, the detector is set at different wavelengths. In a non-limiting example, when the separated AAV genome is detected, the detector is set at an excitation wavelength of about 488 nm and an emission wavelength of about 520 nm, and when the separated AAV capsid protein component is detected, the detector is set at an excitation wavelength of about 488 nm and an emission wavelength of about 600 nm.

The disclosed methods have several advantages over conventional methodologies, including the ability to adapt the disclosed methods to a high-throughput application or a rapid analysis workflow.

Another aspect of the disclosure includes a kit for quantifying an intact adeno-associated virus vector (AAV) genome and quantifying an AAV protein component. The kit may include, but is not limited to, a first buffer comprising a first polymer matrix, a second buffer comprising sodium dodecyl sulfate (SDS) and a second polymer matrix, and instructions for use. The kit may further include at least two capillary electrophoresis (CE) capillaries, at least two CE cartridges comprising at least one capillary, or at least two capillary electrophoresis chips. Depending on the desired analysis, the kit may also include at least one fluorescent dye that binds nucleic acids, at least one fluorescent dye that labels protein, a diluent, a nuclease and/or a proteinase/protease.

### EXAMPLES

### Example 1: Materials, Instrument and Methods

### Materials

The LIF Performance Test Mix (PN: 726022), Nano vials (PN 5043467) and pre-assembled EZ-CE Capillary Cartridge (PN A55625, FIG. 1B) were from SCIEX, Framingham, MA. Nuclease-free water (PN AM9932), SYBR Green II RNA gel stain, 10,000× concentrate in DMSO (PN S7564), 10× DNase I buffer (PN AM8170G) were obtained from Thermo Fisher Scientific, Waltham, MA. benzonase (PN E1014-5KU), 0.5 M EDTA, pH 8.0 (PN E7889-100ML), Transcript RNA markers 0.2-10 kb (PN R7020), Molecular Biology Grade, Amicon Ultra-0.5 centrifugal filter unit with MW cut off of 100 kDa (PN UFC510024) were from Millipore Sigma, St. Louis, MO. The 0.2 μm syringe filter (PN 4612) was from PALL Corporation, Port Washington, NY. Rainin LTS filter tips were from Mettler Toledo, Oakland, CA. QIAquick PCR purification Kit (PN 28104) and Proteinase K (PN 19131) were from Qiagen, Germantown, MD. Packaged AAV8 and AAV formulation buffer (1×PBS with 0.001% Pluronic F68) were from Vigene Biosciences/Charles River Laboratories, Rockville, MD. AAV5 and AAV2 were from SignaGen, Rockville, MD. Firefly Luciferase Control RNA (Fluc RNA, L4561) was from Promega, Madison, WI.

Sample Storage: AAV samples and the Sigma Transcript RNA Markers were aliquoted at 5 to 20 μL upon first thawing and stored at −80° C. freezer to avoid multiple freeze-thaw cycles.

Instrument and software: A PA 800 Plus Pharmaceutical Analysis System or a BioPhase 8800 system equipped with LIF detector and solid-state laser with excitation wavelength at 488 nm and a 520 nm band pass emission filter were from SCIEX (Framingham, MA) and a 600 nm band pass emission filter from Edmund Optics (Barrington, NJ).

The AAV genome Separation Buffer: A commercial separation gel such as ones found in RNA 9000 Purity & Integrity Kit (SCIEX, Framingham, MA) or another type of separation buffer utilized for nucleic acid is utilized, before the sample run, the required aliquot of buffer was warmed up to room temperature and filtered through a 0.2 μm filter. SYBR Green II Gel Stain was added at a 1 to 25,000 dilution. About 7.5 mL of this dye-containing buffer can be used for each set of 8 injections on a PA 800 Plus system. The dye will bind through non-covalent interaction to nucleic acids (single stranded DNA genome, partial genome, RNA/DNA impurities) during the process of CE separation. Similar conditions can be used with BioPhase 8800 system.

Sample Preparation for AAV Genome: For building standard curve: 110 μL of an AAV sample with known titer was serially diluted at 2 fold in 1×PBS from 2.62×10¹³ GC/ml to 1.28×10¹⁰ GC/ml. Samples were kept on ice before extraction of AAV genome. For a test sample, an AAV sample at the volume of 10 to 15 μL was taken out of the freezer, thawed on ice and diluted to 110 μL with 1×PBS, 0.001% pluronic F68 as needed. Alternatively, AAV samples can be treated with Benzonase and Proteinase K prior to dilution and subsequent purification using the QIAquick PCR purification Kit. The benzonase is a non-selective endonuclease that degrades all forms of DNA and RNA to oligonucleotides. Proteinase K can be utilized to release the AAV genome.

AAV capsids were disassembled by adding 550 μL of binding buffer from QIAquick PCR purification Kit to 110 μl of undiluted AAV stock or from each of the dilution point or from the AAV test sample. The AAV genome released from AAV capsids was purified using QIAquick PCR purification Kit following manufacturer's instructions except the columns were washed twice. The AAV genome sample was eluted from the column using 50 μL of nuclease-free water or 10% elution buffer (from the Qiagen kit) diluted with nuclease-free water. After eluting, 10 μL of the AAV genome sample was diluted with 40 μL of water and 50 μL of a sample loading solution. Ten microliters of the diluted, eluted AAV genome sample was heated at 70° C. for 2 minutes, followed by 5 minutes on ice, before transferring to a Nano vial and analyzed on a PA 800 Plus system. For analysis on BioPhase 8800 system, 50 μL to 100 μL of the diluted, eluted AAV genome sample was used.

Preparation of Chromeo P503 Working Solution: A vial of Chromeo P503 dye comes in 1 mg of lyophilized powder. The lyophilized powder was reconstituted by adding 1 mL of methanol or DMSO. Make 10 μL aliquots to prevent contamination due to over-handling. After reconstitution, the dye label can be stored at 2-8° C. for six months according to the manufacturer's instructions.

Sample Dilution Procedure Prior to Labeling: For building standard curve: 100 μL of an AAV sample with known titer was serially diluted at 2 fold in 1×PBS from 2.62×10¹³ GC/ml to 1.60×10⁹ GC/ml. Samples were kept on ice. For a test sample, an AAV sample at the volume of 5 to 15 μl was taken out of the freezer, thawed on ice and diluted to 15 μL with 1×PBS as needed.

Sample Denaturing Procedure Prior to Labeling: One 15 μL of test sample or one 15 μL of aliquot removed from the undiluted AAV stock or from each of the dilution point was mixed with 15 μL of SDS sample buffer and 3 μL of IM Dithiothreitol. The mixture was briefly vortexed for proper mixing and heated to 70° C. in a thermal cycler for 10 minutes and 25° C. for 1 minute. After that, the reaction tubes were removed from the thermal cycler and allowed to cool down to room temperature.

Sample Labeling Procedure: 1.5 μL of 1 mg/mL of Chromeo P503 Labeling Working Solution was added to each reaction tube and briefly vortexed. Once again, the tube was heated to 70° C. in a thermal cycler for 10 minutes and 25° C. for 1 minute. Afterward, the tubes were first allowed to cool to room temperature and then 115.5 μL of DDI water was added. The labeled sample was transferred to the sample vial (for PA 800 Plus system) or sample well on a sample plate (for BioPhase 8800 system) for SDS-CGE-LIF analysis.

Preparation of Buffer Trays and Sample Trays: All solutions were pipetted with filter tips. Each “NF Water” vial was filled with 1.5 mL nuclease-free (NF) water. For analysis on PA 800 Plus system, waste vial was filled with 1 mL NF water. “Genome Sep Buffer” and “TBE Buffer” vials were filled with 1.5 mL of separation buffer containing SYBR Green II Gel Stain or 1×TBE. For analysis on BioPhase 8800 system, the amounts of gel and other solutions were determined by the BioPhase software and added to the reagent inlet and outlet plates and sample outlet plates.

### Example 2: Determination of Full-Capsids % in an AAV Sample

### Using CGE-LIF to Quantify the Amount of Intact AAV Genome in an AAV Sample.

An AAV genome calibration standard was generated by serially diluting the AAV sample with a known titer. The AAV genome was extracted, and the corrected peak area of the intact genome peak by CGE-LIF was determined. This can be performed by mixing 20 μL of each AAV sample with a binding buffer from the QIAquick PCR purification kit and following the manufacturer's instructions in the kit with the exception that the column was washed twice. The AAV genome sample can be eluted from the column using 50 μL of 10× diluted by elution buffer (Buffer EB) from the kit. Before loading onto the instrument for analysis, 10 μL of the eluted AAV genome solution can be mixed with 40 μL of nuclease-free water and 50 μL of sample loading solution, heated at 70° C. for 2 minutes and immediately cooled on ice for 5 minutes. Alternatively, for AAV samples with residual host cell nucleic acids, a longer sample preparation workflow can be used. This longer procedure includes benzonase treatment to degrade small sized impurities outside of AAV capsids, filtration to remove the benzonase and degraded nucleic acids and proteinase K treatment for AAV genome release from the capsids prior to the QIAquick PCR kit purification.

Analysis can be performed on a PA 800 Plus Pharmaceutical analysis system (SCIEX) utilizing a bare fused-silica capillary (50 μm I.D., 30 cm total length, 20 cm effective length) (SCIEX PN A55625, Framingham, MA) using a separation RNA gel buffer such as the one from RNA 9000 Purity & Integrity Kit (SCIEX, Framingham, MA). The capillaries are rinsed sequentially with acid, water, TBE Buffer and the RNA Gel buffer from the kit. Samples were introduced into the inlet of the capillary electrokinetically at −5 kV for 10 s. Separations can be performed using reversed polarity with 300 V/cm electrical field at 25° C. Sample trays are preferably kept at 4° C. to minimize nucleic acid degradation and renaturation. LIF detector was configured with a 488-nm laser with an emission filter of 520 nm. SCIEX Analysis software can be used for data processing.

The corrected peak area was plotted against the AAV titer. As shown in FIGS. 2A and 2B, an AAV genome calibration standard built with AAV samples of known concentrations for genome size analysis used to determine the amount of intact genome present. This is used to generate the first corresponding value, also referred to as Value A. The limit of detection (LOD) values were 1.28×10¹⁰ genome copies (GC)/mL and the limit quantitation (LOQ) values were 2.56×10¹⁰ GC/mL. Value A from genome integrity analysis was 4.17E×10¹² GC/mL for a test sample (12.5 μL of the original sample) was diluted to 110 μL with 1×PBS. The concentration of capsids with intact genome in the original sample was determined to be: 4.17E×10¹² based on the assumption that the AAV samples used for building the standard curve contain only full capsids.

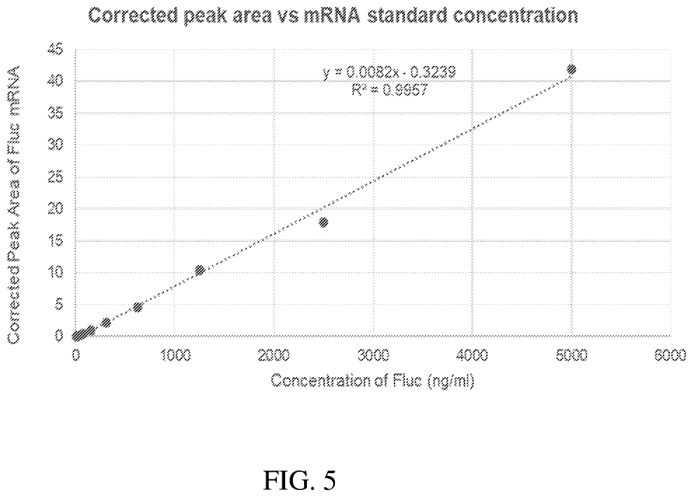

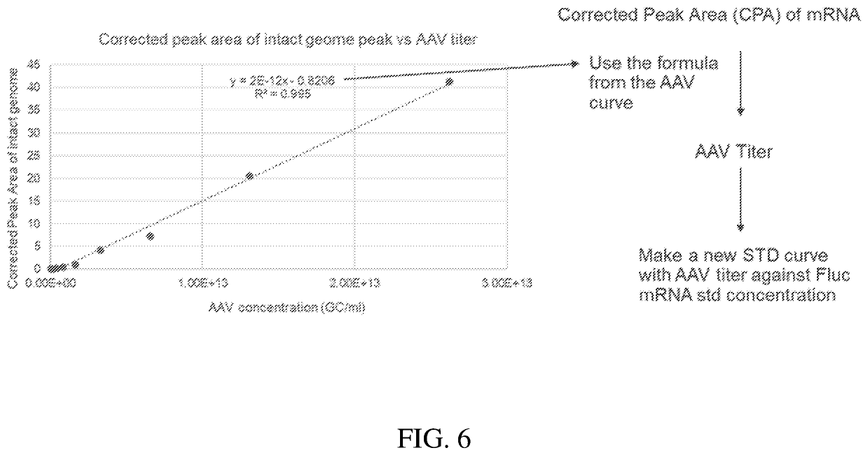

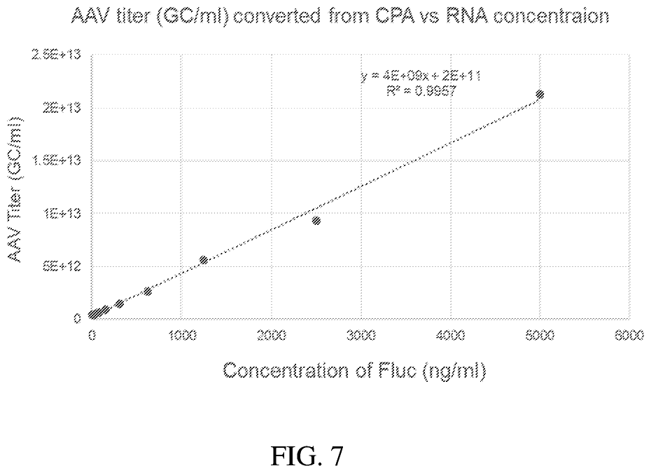

Using AAV sample with known titer for building standard curve requires constant supply of this AAV sample. One alternative could be to use a general nucleic acid or protein to build the standard curve. For example, one can use a nucleic acid standard such as the 1.8 kb firefly mRNA (Fluc mRNA) to build a standard curve to determine the amount of intact genome present in an AAV sample. Since the amount of intact genome in an AAV sample should not be given in terms of Fluc mRNA concentration, the Fluc mRNA concentration needs to be converted to AAV titer in an initial experiment. Serially diluted Fluc mRNA was run with serially diluted AAV sample with known titer in the same separation sequence using the same separation conditions (same separation method, same cartridge and same buffer matrix). Three standard curves were built in this initial experiment. As shown in FIGS. 4-6, the first standard curve was built by plotting the corrected peak area of the Fluc mRNA against Fluc mRNA concentration (FIG. 4). The second standard curve was built by plotting the corrected peak area of the intact genome peak against the concentration or titer of the AAV sample with known titer. Corrected peak area of the Fluc mRNA at each concentration in FIG. 5 was converted to AAV concentration or titer using the linear equation from FIG. 6. An AAV genome calibration standard curve with AAV titer (converted from Fluc mRNA CPA) plotted against Fluc mRNA concentration was then built as shown in FIG. 7. In future experiment, only the Fluc mRNA needs to be run with an AAV test sample. For example, an AAV test sample was run with serially diluted Fluc mRNA. The CPA for the intact genome peak was 0.21. By using the standard curve in FIG. 4, the Fluc mRNA concentration corresponding to this CPA value of 0.21 was determined as 65 ng/ml. Then, the standard curve in FIG. 7 was used to determine the AAV titer to be 4.60×10¹¹ GC/ml when Fluc mRNA concentration was 65 ng/ml. This was close to 4.73×10¹¹ GC/ml, the titer determined from previous experiment with using the standard curve built with AAV sample with known titer. Therefore, this example established the feasibility of using a general nucleic acid like Fluc mRNA for determination of the amount of intact genome in an AAV test sample. This option saves time and money since once the relationship of Fluc mRNA and AAV titer is established in the initial experiment, in future experiments, and the AAV test sample only needs to be run with serially diluted Fluc mRNA which does not need to go through nucleic acid extraction with QIAquick PCR purification kit.

### Using CE-SDS-LIF to Quantify the Amount of Capsid Protein in an AAV Sample.

A CE-SDS Protein Analysis kit (SCIEX PN C30085) can be utilized to quantify the amount of capsid protein. 15 μL of AAV sample, diluted in PBS, can be mixed with 15 μL of SDS sample buffer and 3 μL of IM DTT and incubated at 70° C. for 10 min, followed by adding 1.5 μL of 1 mg/mL Chromeo P503 dye5 and incubated at 70° C. for another 10 min. After cooling the samples down to room temperature, 115.5 μL of deionized water can be added to the mixture . . . . The diluted, prepared sample solution can then be analyzed on a BioPhase 8800 system configured with an LIF detector and solid-state laser with an excitation wavelength of 488 nm and a 600 nm bandpass emission filter. The separations were performed using the pre-assembled BioPhase BFS Capillary Cartridge (PN) (SCIEX Part #5080121) with a 20 cm effective length (30 cm total length). Capillary conditioning can be performed using 0.1 M NaOH rinse for 2 minutes at 80 psi followed by 5 minutes at 20 psi, 0.1 M HCl rinse for 5 minutes at 20 psi, CE-Grade water rinse for 3 minutes at 20 psi and SDS-protein analysis kit buffer rinse for 10 minutes at 80 psi before each run. The applied electric field strength was 500 V/cm for all capillary electrophoresis analyses in reverse polarity mode (anode at the detection side). The samples were electrokinetically injected at 5 kV for 60 seconds in reverse polarity. The BioPhase 8800 software version 1.1 was used for data acquisition and processing.

As described in the methods above, a standard curve was generated by serially diluting the AAV sample with a known titer, labeling them with p503 dye. The corrected peak area of the VP3 capsid peak was determined by CE-SDS-LIF and the corrected peak area was plotted against the AAV titer. As shown in FIGS. 3A and 3B, a standard curve was built with AAV samples of known concentrations for capsid protein analysis were be used to determine the amount of capsid protein present. This is used to generate the second corresponding value, also referred to as Value B. The LOD values were 1.60×10⁹ GC/mL and the LOQ values were 6.40×10⁹ GC/mL. Value B from AAV capsid analysis: 2.65×10¹² GC/mL for the test sample.

Value A (referred to previously) was divided by Value B (referred to previously) to obtain a percentage of full capsids with intact genomes which came out as 157% based on an assumption that the AAV samples used for building the standard curve contain only full capsids with intact genome. It was assumed, based on the available testing information for other lots of the same material, that 53% of capsids contained intact genome (if determined by AUC). Using this number to adjust the percentage of full capsid obtained, the full capsid percentage of the test sample would be: 157%*53%=83%.

While the present disclosure has been described with reference to certain embodiments, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular embodiments disclosed, but that the present disclosure will include all aspects falling within the scope of the appended claims.

All patents, patent applications, publications, and descriptions mentioned above are herein incorporated by reference in their entirety.

1. A method of evaluating at least one adeno-associated virus vector (AAV) sample, the method comprising
loading a first portion of the AAV sample on a first capillary electrophoresis (CE) capillary, wherein the first CE capillary is filled with a first buffer comprising a first polymer matrix;
applying a voltage to the first AAV portion to separate an intact AAV genome from the first portion of the AAV sample;
detecting the separated AAV genome with a detector;
producing an electropherogram comprising corrected peak area of the intact AAV genome and generating a first corresponding set of values;
loading a second portion of the AAV sample on a second capillary electrophoresis (CE) capillary, wherein the second CE capillary is filled with a second buffer comprising sodium dodecyl sulfate (SDS) and a second polymer matrix;
applying a voltage to the second portion of the AAV sample to separate an AAV capsid protein component from the second portion of the AAV sample;
detecting the separated AAV capsid protein component with a detector;
producing an electropherogram comprising corrected peak area of the AAV capsid protein component and generating a second corresponding set of values; and
wherein the first corresponding set of values is used to quantify the intact AAV genome in the AAV sample and the second corresponding set of values is used to quantify the AAV capsid protein component in the AAV sample.

2. The method of claim 1, wherein the AAV sample is selected from the group consisting of wild-type AAVs, recombinant AAVs, AAV serotypes, self-complementary AAVs, and AAV drug products.

3. The method of claim 1 or claim 2, wherein the AAV capsid protein component is selected from the group consisting of VP1, VP2, VP3, and combinations thereof.

4. The method of claim 1 or claim 2, wherein the intact AAV genome is separated from partial genomes and/or impurities in the first portion of the AAV sample.

5. The method of claim 1 or claim 2, wherein the AAV capsid protein component is separated from impurities in the second portion of the AAV sample.

6. The method of claim 5, wherein the AAV capsid protein component is VP3.

7. The method of claim 1 or claim 2, wherein the intact AAV genome concentration is determined by comparing the first corresponding set of values with an intact AAV genome calibration standard.

8. The method of claim 7, wherein the intact AAV genome calibration standard is generated by
loading a AAV genome standard series on a capillary electrophoresis (CE) capillary, wherein the CE capillary is filled with a first buffer comprising a first polymer matrix, the AAV genome standard series comprising at least one concentration of an AAV genome standard;
applying a voltage to the AAV genome standard series to separate intact genome from the AAV genome standard;
detecting the separated intact genome with a detector;
producing an electropherogram comprising corrected peak area of the intact genome and generating a corresponding set of AAV genome standard values; and
wherein the intact AAV genome calibration standard is generated from the corresponding set of AAV genome standard values.

9. The method of claim 8, wherein the AAV genome standard series comprises an AAV sample with a known titer, an RNA sample with known concentration, and/or a single stranded DNA sample with known concentration.

10. The method of claim 9, wherein the AAV genome standard series comprises an RNA sample with a known concentration, and wherein the concentration of the RNA sample is further correlated to an intact genome titer of an AAV genome standard.

11. The method of claim 8, wherein the AAV genome standard series comprises an AAV sample with the known titer and known full %.

12. The method of claim 11, wherein the known full % is obtained using AUC.

13. The method of claim 9, wherein the standard series comprises an AAV sample with the known titer equal or higher than 1×10¹³ GC/ml.

14. The method of claim 11, wherein the known full % of the AAV genome standard is determined using an AAV reference material with full % determined by AUC and with a known titer.

15. The method of claim 13, wherein the known full % of the AAV genome standard is determined using an AAV reference material with full % determined by AUC and with a known titer.

16. The method of claim 7, wherein the concentration of the intact AAV genome is determined by comparing the first corresponding set of values with the intact AAV genome calibration standard and further correcting to account for a presence of genome other than intact genome in the AAV genome standard series.

17. The method of claim 16, wherein further correcting to account for a presence of genome other than intact genome in the AAV genome standard series comprises multiplying by full % determined by AUC.

18. The method of claim 1, wherein the concentration of the AAV capsid protein component is determined by comparing the second corresponding set of values with an AAV capsid protein calibration standard.

19. The method of claim 18, wherein the AAV capsid protein calibration standard is generated by
loading a AAV capsid protein standard series on a second capillary electrophoresis (CE) capillary, wherein the second CE capillary is filled with a second buffer comprising sodium dodecyl sulfate (SDS) and a second polymer matrix, the AAV capsid protein standard series comprising at least one concentration of an AAV capsid protein standard;
applying a voltage to the second standard series to separate AAV capsid protein components from AAV capsid protein standard;
detecting the separated AAV capsid protein components with a detector;
producing an electropherogram comprising corrected peak area of the AAV capsid protein component and generating a corresponding set of AAV capsid standard values; and
wherein the AAV capsid protein calibration standard is generated from the corresponding set of AAV capsid standard values.

20. The method of claim 19, wherein the AAV capsid protein standard series comprises an AAV sample with a known concentration, an AAV sample with a known titer, and/or a protein sample with a known concentration.

21. The method of claim 19, wherein the AAV capsid protein standard series comprises an AAV capsid standard sample with a known titer and known full %.

22. The method of claim 21, wherein the known full % is obtained using AUC.

23. The method of claim 20, wherein the known full % of the AAV capsid standard is determined using an AAV reference material with known full % by AUC and with a known titer.

24. The method of claim 19, wherein the AAV capsid protein standard series comprises at least two different concentration of an AAV capsid protein standard.

25. The method of claim 8, wherein the AAV genome standard and the AAV capsid protein standard are the same.

26. The method of claim 1, wherein the first corresponding set of values is divided by the second of corresponding values to determine a percentage of full AAV capsids with intact AAV genomes.

27. The method of claim 1, wherein the first corresponding set of values is divided by the second of corresponding values and multiplied by the percent of intact genome of the AAV standard to determine a percentage of full AAV capsids with intact AAV genomes in a test sample.

28. The method of claim 27, wherein further correcting to account for a presence of a genome other than intact genome in the AAV genome standard series comprises multiplying by full % determined by AUC.

29. The method of claim 26, wherein an empty/full ratio of the AAV sample is calculated using the full %.

30. The method claim 1, wherein the first CE capillary is housed in a first capillary cartridge and the second CE capillary is housed in a second capillary cartridge.

31. The method of claim 1, where the first buffer comprising a first polymer matrix is different from the second buffer comprising a second polymer matrix.

32. The method of claim 1, wherein the first polymer or second polymer matrix is independently selected from the group consisting of crosslinked polymer, linear polymers, slightly branched polymers, linear polyacrylamide, polyvinylpyrrolidone, polyethylene oxide, polyethylene glycol and dextran.

33. The method of claim 1, wherein the first portion of the AAV sample is denatured or digested prior to loading onto the first CE capillary.

34. The method of claim 33, wherein the first portion of the AAV sample is denatured prior to CE separation.

35. The method of claim 33, wherein the first portion of the AAV sample is digested using at least one endonuclease, protease, peptidase or proteinase.

36. The method of claim 35, wherein the endonuclease is selected from the group consisting of DNase I, benzonase, RNase, and combinations thereof.

37. The method of claim 35, wherein the protease, peptidase or proteinase is Proteinase K.

38. The method of claim 33, wherein the first portion of the AAV sample is purified or enriched prior to loading onto the first CE capillary.

39. The method of claim 38, wherein the first portion of the AAV sample is purified or enriched using spin columns, spin tubes, and/or magnetic beads.

40. The method of claim 1, wherein the first portion of the AAV sample is diluted with a sample solution, water, or combinations thereof prior to loading on the CE capillary.

41. The method of claim 1, further comprising heating the first portion of the AAV sample prior to loading the first portion of the AAV sample on the CE capillary.

42. The method of claim 41, wherein the first portion of the AAV sample is heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C., alternatively at a temperature of about 60° C.

43. The method of claim 41, wherein the first portion of the AAV sample is heated at a temperature of about 70° C.

44. The method of claim 41, wherein the first portion of the AAV sample is heated for at least 2 minutes, alternatively at least 3 minutes, alternatively at least 4 minutes, alternatively at least 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

45. The method of claim 41, wherein the first portion of the AAV sample is heated for at least about 2 minutes.

46. The method of claim 41, further comprising cooling the first portion of the AAV sample after heating.

47. The method of claim 46, wherein the first portion of the AAV sample is cooled using ice or at about 4° C.

48. The method of claim 47, wherein the first portion of the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, or alternatively at least about 60 minutes.

49. The method of claim 1, further comprising adding a fluorescent dye to the first buffer comprising a first polymer matrix, wherein the fluorescent dye binds the AAV sample resulting in fluorescently labeled AAV genome.

50. The method of claim 49, wherein the fluorescent dye is a cyanine-based dye.

51. The method of claim 49, wherein the fluorescent dye is selected from the group consisting of Cy2, Cy3, Cy3B, Cy3.5, Cy5, Cy5.5, Cy7, SYBR Green I, SYBR GOLD, SYBR Green II, PicoGreen, Thiazole orange, and Oxazole yellow.

52. The method of claim 1, wherein the second portion of the AAV sample is denatured or digested prior to loading onto the second CE capillary.

53. The method of claim 1, wherein the second portion of the AAV sample is denatured prior to CE separation.

54. The method of claim 51, wherein the second portion of the AAV sample is denatured using heat, detergent, a reducing agent, sonication, or a combination thereof.

55. The method of claim 54, wherein the second portion of the AAV sample is denatured using heat, SDS, and dithiothreitol.

56. The method of claim 54, wherein the second portion of the AAV sample is heated at a temperature of about alternatively about 50° C., alternatively about 55° C., alternatively about 60° C., alternatively about 65° C., alternatively about 70° C., alternatively about 75° C., alternatively about 80° C., alternatively about 85° C., alternatively about 90° C., alternatively about 95° C.

57. The method of claim 56, wherein the second portion of the AAV sample is heated for at least about 5 minutes, alternatively at least about 10 minutes, alternatively at least about 15 minutes, alternatively at least about 20 minutes, alternatively at least about 25 minutes, alternatively at least about 30 minutes, alternatively at least about 45 minutes, alternatively at least about 60 minutes.

58. The method of claim 54, wherein the second portion of the AAV sample is heated at a temperature of about 70° C.

59. The method of claim 58, wherein the second portion of the AAV sample is heated for at least about 10 minutes.

60. The method of claim 54, wherein the second portion of the AAV sample is cooled to room temperature.

61. The method of 60, wherein the second portion of the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

62. The method of claim 53, further comprising adding a fluorescent dye to the denatured second portion of the AAV sample, wherein the fluorescent dye reacts the AAV sample resulting in fluorescently labeled AAV protein component.

63. The method of claim 62, wherein the fluorescent dye is incubated with the denatured second portion of the AAV sample at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C.

64. The method of claim 63, wherein the fluorescent dye is incubated with the denatured second portion of the AAV sample for at least about 5 minutes, alternatively at least about 10 minutes, alternatively at least about 15 minutes, alternatively at least about 20 minutes, alternatively at least about 25 minutes, alternatively at least about 30 minutes, alternatively at least about 45 minutes, alternatively at least about 60 minutes.

65. The method of claim 62, wherein the fluorescent dye is incubated with the denatured second portion of the AAV sample at a temperature of about 70° C.

66. The method of claim 65, wherein the fluorescent dye is incubated with the denatured second portion of the AAV sample for at least about 10 minutes.

67. The method of claim 62, wherein the fluorescent dye is a cyanine-based dye or a pyrylium-based dye.

68. The method of claim 62, wherein the fluorescent dye is selected from the group consisting of Cy2, Cy3, Cy3B, Cy3.5, Cy5, Cy5.5, Cy7, Rhodmine, Fluorescein, and Fluorescent Chromeo™ Py-Dyes.

69. The method of claim 63, further comprising cooling the second portion of the AAV sample to room temperature after heating.

70. The method of claim 69, wherein the second portion of the AAV sample is cooled for at least about 1 minute, alternatively at least about 2 minutes, alternatively at least about 3 minutes, alternatively at least about 4 minutes, alternatively at least about 5 minutes, alternatively at least about 10 minutes, alternatively at least about 15 minutes, alternatively at least about 20 minutes, alternatively at least about 30 minutes, alternatively at least about 45 minutes, alternatively at least about 60 minutes.

71. The method of claim 1 or claim 2, wherein the second portion of the AAV sample is diluted with a sample solution, water, or combinations thereof prior to loading on the second CE capillary.

72. The method of claim 1, wherein the detector is a UV detector or fluorescence detector.

73. The method of claim 72, wherein the detector is a laser-induced fluorescence (LIF) detector, a lamp-based fluorescence detector, or a native fluorescence detector.

74. The method of claim 72, wherein when the separated AAV genome is detected, the detector is set at an excitation wavelength of about 488 nm and an emission wavelength of about 520 nm, and when the separated AAV protein component is detected, the detector is set at an excitation wavelength of about 488 nm and an emission wavelength of about 600 nm.

75. The method of claim 1 or claim 2, wherein the method is used in a high-throughput application or a rapid analysis workflow.

76. A kit for quantifying an intact adeno-associated virus vector (AAV) genome and quantifying an AAV protein component, the kit comprising:
a first buffer comprising a first polymer matrix,
a second buffer comprising sodium dodecyl sulfate (SDS) and a second polymer matrix,
and instructions for use.

77. The kit of claim 76, wherein the kit further comprises at least two capillary electrophoresis (CE) capillaries or at least two CE cartridge comprising at least one capillary.

78. The kit of claim 76, wherein the kit further comprises at least one fluorescent dye that binds nucleic acids, at least one fluorescent dye that labels protein, a diluent, a nuclease and/or a proteinase/protease.
